(19) United States
(12) Patent Application Publication
(10) Pub. No.: US 2012/0298581 A1
(43) Pub. Date: Nov. 29, 2012

(54) DEVICE AND METHOD FOR DETECTING AN OPERATING STATE OF AN EXTRACORPOREAL BLOOD TREATMENT

(75) Inventors: Wolfgang Wehmeyer, Tuebingon (DE); Andreas Wuepper, Buettelborn (DE)

(73) Assignee: Fresenius Medical Care Deutschland GmbH, Bad Homburg (DE)

(21) Appl. No.: 13/478,362

(22) Filed: May 23, 2012

Related U.S. Application Data
(60) Provisional application No. 61/489,037, filed on May 23, 2011.

(30) Foreign Application Priority Data
May 23, 2011 (DE) 10 2011 102 962.5

(51) Int. Cl. A61M 1/16

(52) U.S. Cl. 210/646; 210/321.65

(57) ABSTRACT

A device (25) and a method for detecting the cause of a deviation from an ideal operating state of an extracorporeal blood treatment, wherein blood to be treated in an extracorporeal blood circulation (9) flows through the blood chamber (3) of a dialyzer (1), subdivided by a semipermeable membrane (2) into a blood chamber (3) and a dialysis fluid chamber (4), and dialysis fluid flows through the dialysis fluid chamber (4). The method includes the following: changing a characteristic (170; 171) of the dialysis fluid upstream from the dialysis fluid chamber (1), measuring a characteristic of the dialysis fluid downstream from the dialysis fluid chamber, determining a dialysance or clearance at a certain point in time during the blood treatment, detecting a deviation in the dialysance or clearance, and detecting the cause of a deviation from an ideal operating state as a function of the certain point in time.

### TECHNICAL FIELD

The invention relates to a device for detecting an operating state of an extracorporeal blood treatment, in particular a device and a method for detecting or determining the cause of a deviation from an ideal operating state of an extracorporeal blood treatment with a blood treatment device, in which the blood to be treated in an extracorporeal blood circulation flows through the blood chamber of a dialyzer, which is subdivided by a semipermeable membrane into the blood chamber and a dialysis fluid chamber, and dialysis fluid flows through the dialysis fluid chamber of the dialyzer in a dialysis fluid circulation.

In addition, the invention relates to a method for detecting or determining a cause or deviation from an ideal operating state or an ideal treatment course of an extracorporeal blood treatment.

### BACKGROUND

Various blood purification methods have become established in kidney replacement therapy; in these treatment methods, blood is freed of ingredients that must be eliminated with the urine, i.e., in an extracorporeal system, ingredients of blood which are eliminated via the kidneys in a healthy person. In hemodialysis a diffuse mass transport of substances present in blood which are normally eliminated in urine takes place through a semipermeable membrane into a dialysis fluid. The mass transport takes place through the semipermeable wall of a dialyzer having a blood chamber connected to an extracorporeal blood circulation and a dialysis fluid chamber connected to a dialysis fluid circulation. The blood chamber and dialysis fluid chamber are separated by the semipermeable membrane. To prevent diffusive loss of electrolytes, which should remain in the blood, the dialysis fluid contains a certain composition of electrolytes in a physiological concentration.

On the other hand, in hemofiltration a convective mass transport occurs through a semipermeable membrane of a filter in which a pressure gradient on the membrane is the driving force for the mass transport. To compensate for a loss of unwanted blood constituents, the electrolytes lost through the membrane must be replaced by a substituate fluid. The substituate fluid may be added upstream or downstream from the filter at an injection site. In the first case we speak of predilution and in the second case postdilution. A combination of a convective transport and a diffusive transport is known as hemodiafiltration. When speaking of dialysis or a dialysis treatment in the context of this patent application, this should be understood to refer to a purely diffusive dialysis or hemodiafiltration.

In monitoring an extracorporeal blood treatment, the monitoring of the blood cleaning performance or clearance is important. The clearance K is defined as the proportion of the blood flow through the blood chamber which is purified completely of toxins, in particular urea. In practice instead of the clearance, the dialysance is determined in which the permeability of the filter membrane for electrolytes present in the dialysis fluid is measured. To do so, the concentration of one or more electrolytes in the dialysis fluid in the dialysis fluid circulation upstream from the dialysis fluid chamber is modified and the resulting change in concentration downstream from the dialysis fluid chamber is determined. The modified concentration of one or more electrolytes upstream and downstream from the dialysis fluid chamber influences the conductivity of the dialysis fluid and is measured by means of corresponding conductivity sensors upstream and downstream from the dialysis fluid chamber. Such a method for determining the clearance during the ongoing treatment is referred to as online clearance monitoring and is disclosed in European patent EP 0 911 043 of the present applicant, the disclosure content thereof being fully incorporated into the present patent application. A commercial method based on this principle is distributed by the company Fresenius Medical Care Deutschland GmbH under the name OCM (Online Clearance Monitor).

The dialysance or clearance depends on a number of factors such as the type of filter, the effective area of the filter membrane, the permeability of the filter membrane, the flow rate of blood, the flow rate of the dialysis fluid. When taken separately, the determination of the dialysance or clearance is therefore not a relevant measure for detecting or determining an operating situation or an operating state of an extracorporeal blood treatment.

There may be a deviation from an ideal operating state of the extracorporeal blood treatment due to the fact that blood diverted from the venous branch of the extracorporeal blood circulation into the patient's vascular access goes from there directly into the arterial branch of the extracorporeal blood circulation. This phenomenon is known as fistula recirculation since as a fistula serves as the vascular access. A corresponding phenomenon in which blood purified in the extracorporeal blood circulation is directed through the patient's cardiopulmonary circulation and from there into the arterial branch of the extracorporeal blood circulation is known as cardiopulmonary recirculation.

No methods for determining recirculation are based on influencing physical or chemical characteristics in the venous branch of the extracorporeal blood circulation and subsequently measuring the physical or chemical characteristic in the arterial branch of the extracorporeal blood circulation. For example, the blood temperature in the venous branch of the blood circulation may be altered by either drawing heat directly or by lowering the temperature of the dialysis fluid for a short period of time, whereupon the drop in temperature on the dialysate side affects the blood side in a predetermined manner by way of the semipermeable membrane. To measure the effect of the drop in temperature on the arterial branch of the blood circulation, a temperature pickup may be provided in the arterial blood circulation. In general to determine the recirculation, a device for altering a physical or chemical property of the blood flowing in the venous branch of the extracorporeal blood circulation as well as a measurement pickup for recording a measured variable of the blood flowing back into the arterial branch of the extracorporeal blood circulation must be provided. However, these measures necessitate an increased equipment complexity of the blood treatment device.

Therefore the object of the present invention is to make available a device and a method for detecting or determining an operating state in an extracorporeal blood treatment such that at least one of the aforementioned problems will be overcome.

### SUMMARY

In agreement with the teaching of the invention, this object is achieved by a device for detecting the cause of a deviation from an ideal operating state or an ideal treatment method in an extracorporeal blood treatment. In the extracorporeal blood treatment, the blood to be treated flows through a blood chamber of a dialyzer, which is subdivided by a semipermeable membrane into the blood chamber and a dialysis fluid chamber in an extracorporeal blood circulation and dialysis fluid in a dialysis fluid circulation flows through the dialysis fluid chamber of the dialyzer.

The device for detecting the cause of a deviation from an ideal operating state and/or an ideal treatment course in an extracorporeal blood treatment contains means for altering a physical or chemical characteristic of the dialysis fluid upstream from the dialysis fluid chamber during the blood treatment, measurement means for measuring a physical or chemical characteristic of the dialysis fluid downstream from the dialysis fluid chamber, a device for determining a value of the dialysance or clearance from a change in a measured physical or chemical characteristic at a certain point in time during the blood treatment, a device for detecting a deviation in the value determined for the dialysance or clearance from a reference value of the dialysance or clearance which represents an ideal or typical or complication-free course of treatment and an evaluation unit for detecting the cause of a deviation from an ideal operating state as a function of a certain point in time.

According to one aspect, in agreement with the teaching of the present invention, the dialysance or clearance is determined with the following means as follows: means for altering a physical or chemical characteristic of the blood upstream from the blood chamber during the blood treatment, measurement means for measuring a physical or chemical characteristic of the blood downstream from the blood chamber, a device for determining a value of the dialysance or clearance from a change in the measured physical or chemical characteristic at a certain point in time during the blood treatment.

In agreement with the teaching of the invention, this object is additionally achieved by a method for detecting the cause of a deviation from an ideal operating state or an ideal treatment course in an extracorporeal blood treatment. In the extracorporeal blood treatment, the blood to be treated in an extracorporeal circulation flows through a blood chamber of a dialyzer, which is subdivided by a semipermeable membrane into the blood chamber and a dialysis fluid chamber, and dialysis fluid flows through the dialysis fluid chamber of the dialyzer in a dialysis fluid circulation. This method comprises the following steps:
modifying a physical or chemical variable of the dialysis fluid upstream from the dialysis fluid chamber during the blood treatment,
measuring a physical or chemical characteristic of the dialysis fluid downstream from the dialysis fluid chamber,
determining a value of the dialysance or clearance from a change in the measured physical or chemical characteristic at a certain point in time during the blood treatment,
determining a deviation in the measured value of the dialysance or clearance from a reference value of the dialysance or clearance representing an ideal or typical or uncomplicated course of treatment and
detecting the cause of a deviation from an ideal operating state of the blood treatment as a function of the point in time determined.

According to another aspect, in agreement with the teaching of the present invention, the dialysance or clearance is determined as follows:
modifying a physical or chemical variable of the blood upstream from the blood chamber during blood treatment,
measuring a physical or chemical characteristic of the blood downstream from the blood chamber and
determining a value of the dialysance or clearance from a change in the measured physical or chemical characteristic at a certain point in time during the blood treatment.

According to another aspect in agreement with the teaching of the present invention another device for detecting the cause of a deviation from an ideal operating state or an ideal treatment course in an extracorporeal blood treatment is provided. This additional aspect also starts from an extracorporeal blood treatment in which the blood to be treated in an extracorporeal blood circulation flows through a blood chamber of a dialyzer, which is subdivided by a semipermeable membrane into the blood chamber and a dialysis fluid chamber, and dialysis fluid in a dialysis fluid circulation flows through the dialysis fluid chamber of the dialyzer.

The device for detecting the cause of a deviation from a ideal operating state and/or an ideal treatment course in an extracorporeal blood treatment contains measurement means for measuring a physical or chemical characteristic of the dialysis fluid downstream from the dialysis fluid chamber, wherein the characteristic variable is suitable for determining the concentration of a substance, for example, urea, which is not present in the dialysis fluid upstream from the dialysis fluid chamber or is present only in a negligible concentration, a device for determining a value of the concentration of this substance in the dialysis fluid or in the blood at a certain point in time during the blood treatment, a device for detecting a deviation in the value determined for the concentration of the substance from a reference value of the concentration representing an ideal or typical treatment course or from an ideal concentration profile over time during the treatment and an evaluation unit for detecting the cause of a deviation from an ideal operating state as a function of a certain point in time.

In agreement with the additional aspect of the invention, a method for detecting the cause of a deviation from an ideal operating state or an ideal treatment course is provided. In the extracorporeal blood treatment, the blood to be treated in an extracorporeal circulation flows through a blood chamber of a dialyzer divided by a semipermeable membrane into the blood chamber and a dialysis fluid chamber, and dialysis fluid flows through the dialysis fluid chamber of the dialyzer in a dialysis fluid circulation.

This method comprises the following steps:
measuring a physical or chemical characteristic of the dialysis fluid downstream from the dialysis fluid chamber, this characteristic being suitable for determining the concentration of a substance such as urea which is not present in the dialysis fluid upstream from the dialysis fluid chamber or is present only in a negligible concentration,
determining a value of the concentration of the substance in the dialysis fluid or in the blood at a certain point in time during the blood treatment,
detecting a deviation in the value determined for the concentration of the substance from a reference value of the concentration representing an ideal or typical treatment course or from an ideal concentration profile over time during the treatment and
detecting a cause of the deviation from an ideal operating state as a function of the point in time determined.

In addition, the object of the present invention is represented by a blood treatment device according to claim 11 and a computer program product according to claim 22. Advantageous embodiments are characterized in the dependent claims.

### BRIEF DESCRIPTION OF THE DRAWINGS

Additional details and advantages of the invention are described in greater detail on the basis of an exemplary embodiment depicted in the drawing, in which:

FIG. 1 shows a block diagram of a dialyzer with a device for detecting the cause of a deviation from an ideal operating state,

FIG. 2a shows a diagram of a dialyzer with the inlet and outlet and symbols used in the mathematical derivations,

FIG. 2b shows the course over time of the measured electrolyte concentration upstream and downstream from the dialyzer after a concentration bolus,

FIG. 3 shows the course over time of a measured dialysance and the course of the dialysance over time in an ideal treatment course,

FIG. 4 shows a flow chart of a method for detecting the cause of a deviation from an ideal operating state in an extracorporeal blood treatment,

FIG. 5 shows a flow chart of a method for detecting the cause of a deviation from an ideal operating state of the blood treatment as a function of a certain point in time, and

FIG. 6 shows a block diagram of a network of dialysis machines for performing a method for detecting the cause of the deviation from an ideal operating state.

### DETAILED DESCRIPTION OF THE DRAWINGS

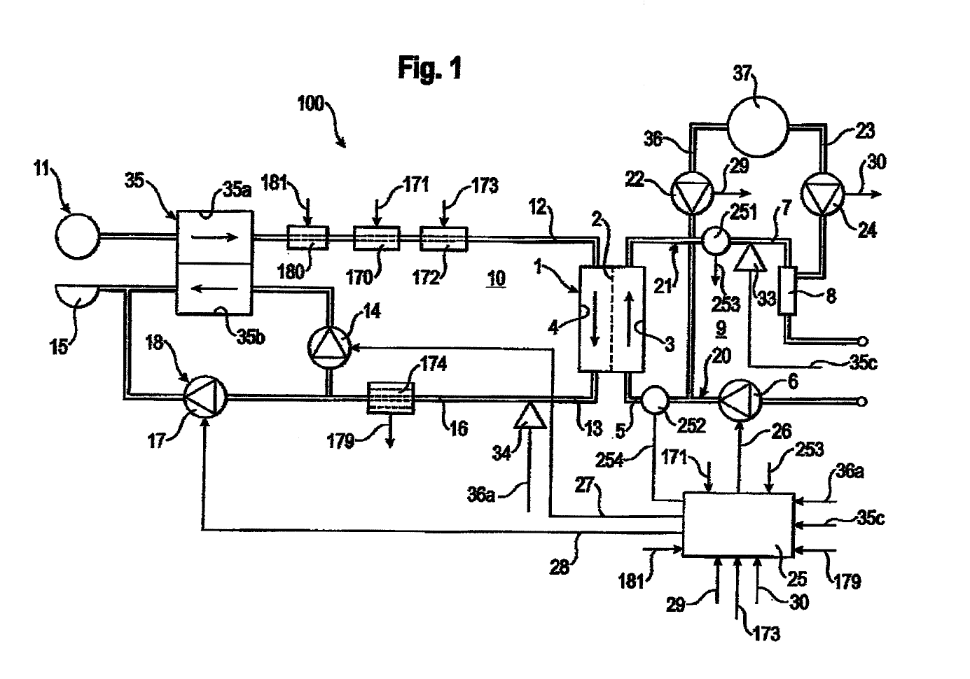

FIG. 1 shows a dialysis device 100 with a control and monitoring unit 25 for detecting the cause of a deviation from an ideal operating state or an ideal treatment course in an extracorporeal blood treatment.

The dialysis device 100 consists essentially of a blood circulation 9, a dialysis fluid circulation 10 and a dialyzer 1, which is divided by a membrane 2 into a blood chamber 3 of the blood circulation 9 and a dialysis fluid chamber 4 of the dialysis fluid circulation 10. The extracorporeal blood circulation 9 consists of an arterial branch 20 with an arterial blood line 5 carrying blood taken from the patient to the blood chamber 3. The arterial branch 20 typically contains a blood pump 6, which may be designed as a roller pump, for example, and can be controlled via a control line 26 from the control and evaluation unit 25. Blood purified in the blood chamber 3 is returned to the patient via a venous line 7 of a venous branch 21. The venous line typically contains a drip chamber 8 for degassing the blood before it is returned to the patient.

In the case of a hemodiafiltration treatment, the extracorporeal blood circulation is connected to a source for substituate liquid 37, from which substituate fluid may be added to the drip chamber 8 via a venous substituate line 23 by means of a venous substituate pump 24 (postdilution). To do so, the venous substituate pump 24 may be connected to the control unit 25 via a control line 30. Additionally or alternatively, an arterial substituate line 36 may also be provided through which substituate fluid can be added to the arterial blood circulation 20 by means of an arterial substituate pump 22 (predilution). To control this, the arterial substituate pump 22 may be connected to the control unit 25 via a control line 29.

The dialysis fluid circulation contains a dialysis fluid inlet line 12 which is connected to the dialysis fluid source 11 and which brings dialysis fluid to the dialysis fluid chamber 4 of the dialyzer 1. Spent dialysis fluid is discharged into a drain 15 via a dialysis fluid outlet line with sections 13, 16. The dialysis fluid in the dialysis fluid circulation is circulated through a dialysis fluid pump 14. A balancing chamber pump 35 balances between the fresh dialysis fluid delivered through the first balancing chamber 35a and the spent dialysis fluid flowing through the second balancing chamber 35b. In the case of hemodiafiltration, an ultrafiltration pump 17 pumps spent dialysis fluids past the balancing chamber. The quantity of fluid pumped out by the diafiltration pump may be used as a measure of the fluid removed through the membrane 2. The dialysis fluid pump 14 can be controlled by the control and evaluation unit 25 via a control line 27 and the ultrafiltration pump 17 can be controlled by the control and evaluation unit 25 via a control line 28.

The dialysis fluid inlet line contains a bolus-creating means 170 with the help of which a concentration bolus of an electrolyte, for example, whose dialysance is to be determined can be created. The bolus-creating means 170 is connected via a data line 171 to the control and evaluating unit 25, which can control the bolus-creating means via the data line 171, so that a concentration bolus is created. The concentration bolus may be a positive concentration bolus, in which a predetermined amount of a concentrate, for example, an electrolyte is added to the dialysis fluid inlet line or a negative concentration bolus in the dialysis fluid in which a predetermined amount of a dilution fluid such as distilled water is added to the dialysis fluid. The bolus-creating means may be designed to create a plurality of liquid boli at successive points in time. The concentration profile of the liquid bolus may be determined either in a calibration step before the actual use of the concentration bolus when adequate reproducibility of the concentration profile can be ensured. As an alternative the concentration profile can be measured with a concentration measurement sensor 172 arranged upstream from the dialysis fluid chamber and downstream from the bolus-generating means 170 in inlet line of the dialysis fluid during the administration of the concentration bolus. For example, a conductivity meter may be used as the concentration measurement sensor 172. The concentration measurement sensor 172 is connected by a data line 173 to the control and evaluating unit 25, which can record the course of the bolus concentration upstream from the dialyzer during the addition of the bolus, recording it in a corresponding memory unit of the control and evaluation unit 25. Alternatively, a criterion for the concentration bolus obtained previously in a calibration step may be stored in a memory unit of the control and evaluation unit 25.

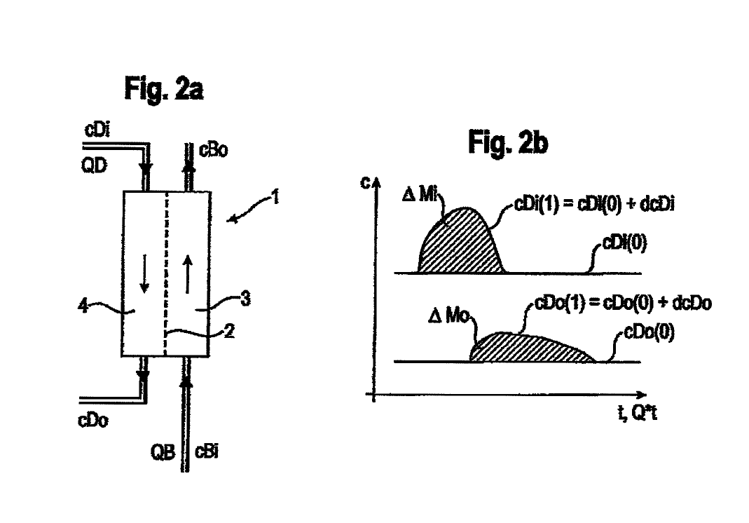

As shown in FIG. 2b in greater detail, the profile of the concentration bolus changes as the concentration bolus is passed through the fluid chamber 4 of the dialyzer 1. The concentration bolus of the dialysis fluid passed through the fluid chamber 4 can be measured with the concentration measurement sensor 174 arranged downstream from the dialysis fluid chamber 4 in the outlet line of the dialysis fluid. The concentration measurement sensor 174 may be designed as a conductivity sensor and is connected by a data line 179 to the control and evaluation unit 25 which can record the course of the bolus concentration after addition of the bolus downstream from the dialyzer in a corresponding memory unit. From the comparison of the bolus concentration upstream from the dialyzer and the bolus concentration downstream from the dialyzer, the control and evaluation unit 25 can determine the dialysance of the concentrate or electrolyte and thus indirectly also the clearance by using the formalism explained below in conjunction with FIGS. 2a and 2b.

In an advantageous embodiment, the dialysis device 1 comprises measurement means for determining the recirculation, which is based on a temperature bolus. Recirculation should be understood here to refer to the return of added blood back to the patient through the venous line into the arterial line. Such a measurement means for determining the recirculation typically comprises a bolus-generating means 180 for generating a temperature bolus in the dialysis fluid inlet line 12, which is connected via a control line 181 to the control and evaluation unit 25. The temperature bolus in the dialysis fluid inlet line is transferred by the dialyzer 1 to the blood circulation 9, where it can be measured with a temperature sensor 251, which is connected via a data line 253 to the control and evaluation unit 25. The temperature bolus in the venous line 21 propagates along the recirculation pathways in the patient's body and can then be measured in the arterial blood line 20 by an arterial temperature sensor 252. Measurement results of the arterial temperature sensor 252 can be transmitted over the data line 254 to the control and evaluation unit 25.

By comparing the temperature bolus measured arterially and that measured venously, it is possible to determine the recirculation of the blood returned via the venous line 7 back into the arterial side. A corresponding method is disclosed in WO 2006/072271 and is included explicitly in the disclosure content of the present patent application. In the preferred embodiment mentioned, the control and evaluation unit 25 is configured to perform this measurement method.

In another preferred embodiment, the dialysis device 1 contains measurement means for determining clotting of the semipermeable membrane. The dialysis device 1 therefore has a first sound pickup 34 in the dialysis fluid circulation and a second sound pickup 33 in the extracorporeal blood circulation. The first and second sound pickups are suitable for picking up a pressure pulse generated by a pressure pulse-generating means in the dialysis fluid circulation or in the blood circulation. A pressure pulse-generating means in the blood circulation would be, for example, the blood pump 6, a pressure pulse-generating means in the dialysis fluid circulation would be, for example, the dialysis fluid pump 14 or the ultrafiltration pump 17.

By comparing the pressure pulses recorded by the first and second sound pickups 33 and 34, it is possible to determine the attenuation of sound waves in the dialyzer and thereby infer the sound attenuation by the dialyzer membrane. This in turn allows an inference regarding the flow resistance of the membrane, the condition of the membrane pores and possible clotting of the membrane.

Such a method for determining the condition of the dialyzer membrane by comparing pressure measurements in the dialysis fluid branch and in the extracorporeal blood circulation is disclosed in WO 2008/135193 and is included explicitly in the disclosure content of the present patent application.

The pressure sensor 33 and 34 are connected to the control and evaluation unit 25 via data lines 35c and/or 36a which perform the corresponding evaluations for determination of the attenuation of sound in the dialyzer and for determining clotting of the membrane pores.

When the dialysis fluid pump 14 is designed as an occlusion pump, it is especially suitable as a pressure pulse-generating means. In this case it generates a series of pressure pulses or oscillating pulses. These oscillating pulses may be picked up by means of the sound pickup 33 on the side of the extracorporeal blood circulation, transmitted via the data line 35c to the control and evaluation unit 25 and evaluated there.

The pressure pulses generated by the blood pump 6 are transmitted over the arterial blood line to the blood chamber 3 of the dialyzer 1 and from there through the membrane 2 of the dialyzer 1 to the dialysis fluid circulation, where they can be picked up with the sound pickup 34, transmitted over the data line 36a to the control and evaluation unit and then evaluated.

This evaluation may be performed, for example, by subjecting the signal picked up by the pressure sensor 33 and the signal picked up by the sensor 34 to a spectral analysis and comparing the spectra of the signal picked up by the pressure sensor 33 and the signal picked up by the pressure sensor 34 to one another. To do so, the control and evaluation unit may contain a Fourier analysis device, for example, which breaks down the signal from the pressure sensor 33 and the signal from the pressure sensor 34 into its spectral components. A comparison of the amplitude spectra of the signal of pressure sensor 33 and of the signal of pressure sensor 34 allows an inference regarding the frequency-dependent attenuation of the oscillating pulses by the dialyzer membrane 2. If this attenuation is greater than a threshold value, which can be determined experimentally, this allows the conclusion that the dialyzer membrane is covered with a secondary membrane, i.e., so-called clotting has occurred.

FIG. 2a shows a dialyzer 1 with a dialysis fluid chamber 4 of a dialysis fluid circulation which is separated by a membrane 2 from the blood chamber 3 of a blood circulation. The flow of dialysis fluid is referred to using the abbreviation QD and the blood flow is referred to with the abbreviation QB; cBi is a concentration of the concentrate of a certain electrolyte at the blood inlet, cBo is the corresponding concentration of the blood outlet; cDi is the concentration of the concentrate, e.g., of a certain electrolyte in the dialysis fluid at the dialysate inlet and cDo is the corresponding concentration in the dialysis fluid at the dialysate outlet.

FIG. 2b shows the course of the change in the dialysis fluid inlet and outlet concentration cDi and cDo as a function of the time. It can be seen clearly that the concentrate bolus occurs at the outlet of the dialyzer with a time lag. The amplitude of the concentrate bolus is lower at the outlet of the dialyzer than that the inlet of the dialyzer. Only the portion of the concentrate bolus which cannot be attributed to a basic concentration is to be taken into account. The basic concentration on the inlet side of the dialyzer is referred to as cDi(0), and the basic concentration on the outlet side of the dialyzer is cDo(0). The total concentration of the concentrate on the inlet side of the dialyzer is referred to as cDi(1), and the total concentration of the concentrate on the outlet side of the dialyzer is cDo(1). The part of the concentrate bolus to be taken into account, i.e., the portion that is not attributable to a basic concentration is referred to as dcDi for the inlet side of the dialyzer and dcDo for the outlet side of the dialyzer. To determine the basic concentration, measured values upstream and/or downstream from the bolus may be used. The control and evaluation unit 190 has a computation unit, which calculates the two variables ΔMi and ΔMo according to the following equations from the plot of the dialysis fluid concentration as a function of time upstream or downstream from the dialyzer and the dialysis fluid rate QD:

ΔMi=QD*∫dcDi dt (1)

ΔMo=QD*∫dcDo dt (2)

The dialysance D is calculated from the variables ΔMi and ΔMo according to the following equation:

$$D = QD * \frac{Δ Mi - Δ Mo}{Δ Mi} . \qquad ( 3 )$$

During the dialysis treatment, the control and evaluation unit 190 continuously performs measurements to determine the dialysance or clearance at certain intervals of time, for example, at intervals of 20-30 minutes.

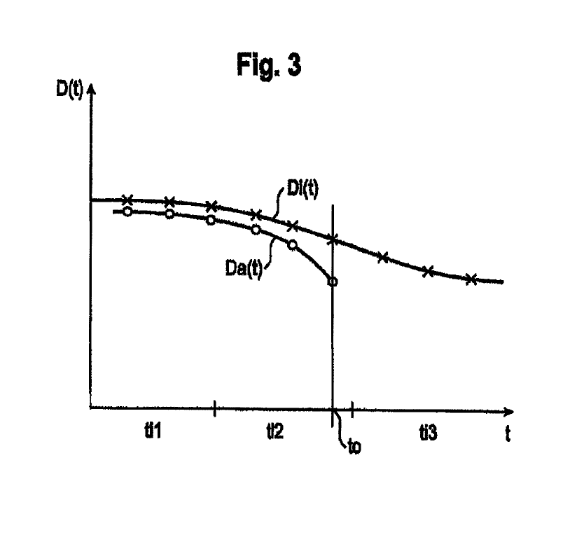

FIG. 3 shows the course of the dialysance Di(t) as a function of time in relation to a starting point in time of the dialysis treatment with an ideal or uncomplicated course of the dialysis treatment, the ideal course of the dialysance. The ideal sequence over time can be obtained by averaging from uncomplicated previous treatments or by averaging from uncomplicated past treatments, corrected or compensated by a factor or percentage which takes into account the treatment parameters of the ongoing treatment. Such parameters of ongoing treatment may include, for example, the instantaneous blood flow rate, the instantaneous dialysis fluid flow rate, the instantaneous ultrafiltration rate, the rate of substituate fluid flow, the location where the substituate fluid is added, i.e., predilution or postdilution, the type of dialyzer or the connection of the connectors for direct current or countercurrent operation.

Thus, for example, starting from the known treatment parameters, a theoretical estimate of the clearance can be given and the course of the dialysance over time can then be standardized to this theoretical estimate of the clearance.

The change in dialysance during the dialysis treatment according to the ideal course plotted over time represents reproducible effects, which are essentially the same with each dialysis treatment of a certain patient such as aging of the pump segment, deposits on and contamination of the membrane and the change in the water content of the blood due to ultrafiltration and a restricted refilling as well as cardiopulmonary recirculation. The aging of the pump segment is thus attributed to the forces exerted by the roller pump. Deposits on the membrane may be protein deposits or the formation of a second layer (so-called second layer deposition). The increase in the hematocrit and the total protein content of the blood lead to a reduction in the blood water content and a reduction in the measured dialysance or clearance. The increase in hematocrit also leads to a reduced blood pressure, which then leads to a reduced cardiac output. This in turn leads to increased cardiopulmonary recirculation.

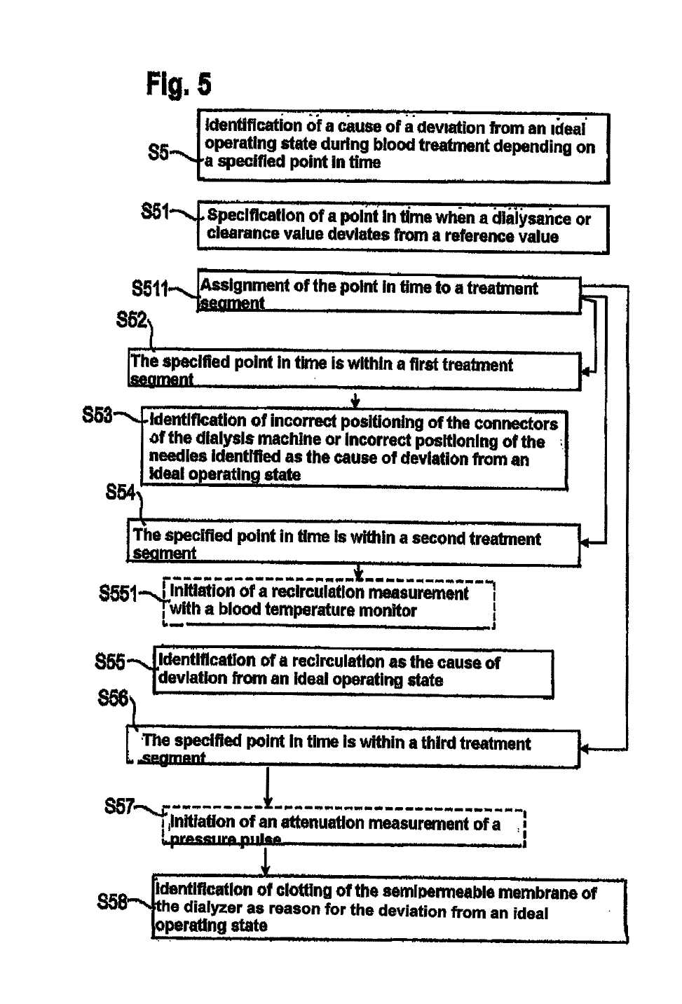

Da(t) refers to measured values for the dialysance or clearance determined during an ongoing dialysis treatment at different points in time after the start of the dialysis treatment. At a point in time to the dialysance or clearance measured currently is compared with the ideal curve of the dialysance. If a significant deviation is found, then the point in time to at which the significant deviation is detected to a current treatment segment is allocated. To do so, the time axis is divided into various treatment segments, namely here a first, second and third treatment segment ti1, ti2 and ti3. This shows an allocation of the point in time to the second treatment segment ti2. The meaning of the first, second and third treatment segments is explained in greater detail below in conjunction with the description of FIG. 5.

If the ideal or uncomplicated course of the dialysance Di(t) has been determined under treatment parameters which deviate from the treatment parameters of the ongoing treatment, then the ideal or uncomplicated course of the dialysance Di(t) can be compared with the value Da(t) during an ongoing dialysis treatment if both Di(t) and Da(t) are standardized to a theoretical value of the clearance which takes into account the respective treatment parameters.

Thus, starting from the mass transfer coefficient KoA, the following equation can be given for the flow rate of the dialysate in the dialyzer Q_{Di} and the blood-water flow rate in the dialyzer Q_{Bi} in the case of hemofiltration for a theoretical estimate or a theoretical value K_{HD} of the clearance:

$${K}_{HD} = {Q}_{bw} * \frac{[ exp ( KoA * ( \frac{1}{{Q}_{Bi}} - \frac{1}{{Q}_{Di}} ) ) ] - 1}{exp ( KoA * ( \frac{1}{{Q}_{Bi}} - \frac{1}{{Q}_{Di}} ) - \frac{{Q}_{Bi}}{{Q}_{Di}} )} \qquad ( equation 1 )$$

In the case of hemodiafiltration, the dialysate flow rate from the dialyzer Q_{Do}, the blood-water flow rate from the dialyzer and the rate of the amount of ultrafiltrate Q_{F} withdrawn via the dialyzer membrane must additionally be taken into account for the theoretical estimate or the theoretical value of the clearance K_{HDF}.

If the following auxiliary variables are defined:

$$f = \frac{KoA}{{Q}_{F}} - \frac{1}{{e}^{\frac{{Q}_{F}}{KoA}} - 1} \qquad ( equation 2 )$$

p=KoA+(1−f)*Q_{F} (equation 3)

and

$$Z = {( 1 - \frac{{Q}_{F}}{{Q}_{Bi}} )}^{\frac{p}{{Q}_{F}} - 1} * {( 1 - \frac{{Q}_{F}}{{Q}_{Do}} )}^{- ( \frac{p}{{Q}_{F}} - 1 )} \qquad ( equation 4 )$$

the following theoretical estimate or the following theoretical value for the clearance case K_{HDF} in the case of ultrafiltration can be given:

$${K}_{HDF} = {Q}_{Bi} * \frac{1 - \frac{{Q}_{Do}}{{Q}_{Di}} * \frac{{Q}_{Bo}}{{Q}_{Bi}} * Z}{1 - \frac{{Q}_{Bo}}{{Q}_{Di}} * Z} \qquad ( equation 5 )$$

With the help of a theoretical estimate or a theoretical value for the clearance in hemodialysis K_{HD} or the clearance in hemofiltration K_{HDF}, the ideal or uncomplicated course of dialysance Di(t) as well as the course of the dialysance Da(t) measured during the ongoing treatment can be standardized to the theoretical clearance K_{HD} and/or K_{HDF}.

The determination of whether there is a significant deviation in the dialysance Da(t) measured during the ongoing treatment may be performed in the following manner.

If the treatment parameters of the current treatment deviate from past uncomplicated treatments, then first the standardized value of the deviation

$$\frac{{D}_{a} ( t ) - {K}_{HD}}{{K}_{HD}}$$

(for hemodialysis) or

$$\frac{{D}_{a} ( t ) - {K}_{HDF}}{{K}_{HDF}}$$

(for hemofiltration) between the theoretical clearance and the measured dialysance or clearance is calculated for the ideal uncomplicated treatment. As an additional step the standardized deviation between the theoretical clearance and the measured dialysance

$$\frac{{D}_{i} ( t ) - {K}_{HD}}{{K}_{HD}}$$

$$or$$

$$\frac{{D}_{i} ( t ) - {K}_{HDF}}{{K}_{HDF}}$$

is calculated for the present treatment.

If the present deviation differs significantly from the ideal uncomplicated deviation, e.g., by more than 10% (e.g.,

$$\vert \frac{{D}_{i} ( t ) - {K}_{HD}}{{K}_{HD}} \vert = 5 \% , \vert \frac{{D}_{a} ( t ) - {K}_{HD}}{{K}_{HD}} \vert \geq 15 \%$$

then the existence of a problem is concluded.

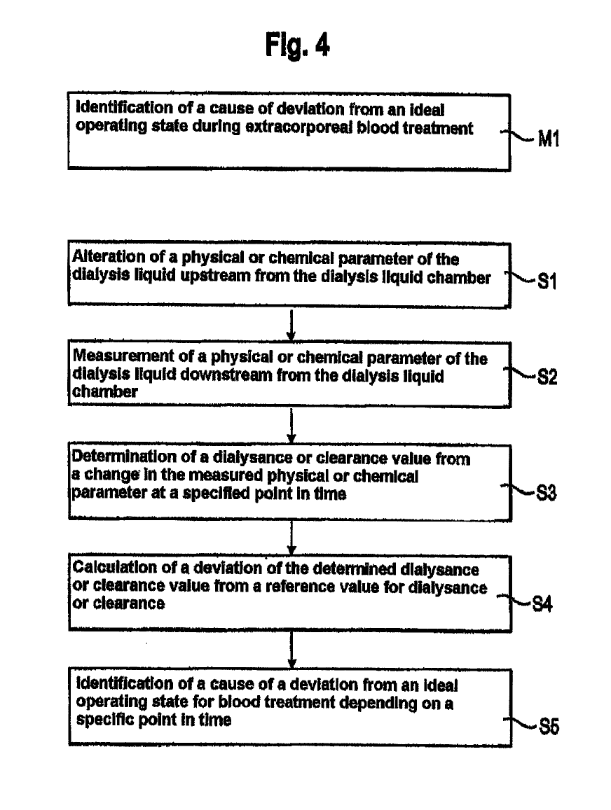

FIG. 4 shows a method M1 for detecting a deviation from an ideal operating state in an extracorporeal blood treatment. The ideal operating state corresponds to a course of the treatment in which no complications or special events occur.

In a step S1 a physical or chemical characteristic of the dialysis fluid upstream from the dialysis fluid chamber is modified. The physical or chemical variable may be an electrolyte concentration in the dialysis fluid upstream from the dialysis fluid chamber. The electrolyte concentration may be obtained by adding a predetermined quantity of a concentrate or electrolyte to the dialysis fluid or from a preparation of the dialysis fluid using a predetermined quantity of an electrolyte. The concentration bolus thereby created can be measured in its course over time by determining the electrolyte concentration upstream from the dialysis fluid chamber by means of a conductivity measurement if, for the electrolyte determined, the ratio between the electrolyte concentration and the conductivity and optionally the concentration of additional electrolytes is already known.

In a second step, the physical or chemical characteristic of the dialysis fluid is determined in its course over time downstream from the dialysis fluid chamber in order to determine a concentration profile downstream from the dialyzer. In the case of the electrolyte concentration as a physical or chemical characteristic, this can be done with the help of a conductivity cell situated downstream from the dialyzer in the dialysis fluid circulation.

In a third step S3, the dialysance or clearance is determined from the change in the measured physical variable at a certain point in time. For example, the dialysance or clearance may be determined from the concentration bolus and the course of the physical or chemical characteristic over time. This may be done, for example, by comparing the bolus profile with the concentration profile measured downstream from the dialyzer by using the formalism of FIGS. 2a and 2b. The point in time when the dialysance or clearance is determined may then be any desired predetermined point in time within the measurement interval.

Next in a fourth step S4 a deviation of the certain value of the dialysance or clearance from a predetermined reference value of the dialysance or clearance is determined, representing an ideal or uncomplicated course of treatment.

The fourth step may include a step of determining whether the deviation thus found is a significant deviation. The deviation may be such that the measured dialysance or clearance is higher or lower than the dialysance or clearance, corresponding to an ideal course of treatment, whether the deviation represents a significant deviation can be determined by comparing the difference thereby found between the measured and ideal clearance or dialysance with a predetermined threshold value or as a relative percentage or standardized deviation. If the deviation is below the predetermined threshold value, then an existing deviation is classified as an insignificant deviation. If the deviation is higher than the predetermined threshold value, then the deviation is classified as a significant deviation. A significant deviation may be, for example, a deviation of more than 10% above or below the predetermined threshold or more than 15% above or below the predetermined threshold value. The dialysance or clearance as a function of a percentage deviation may be classified as significant, so that the percentage at which a deviation is significant is determined as a function of the treatment step.

If the deviation is significant, then an error message may be displayed on the dialysis machine, reporting a significant deviation in the dialysance or clearance. Alternatively or additionally a significant deviation may be recorded in a patient data record, for example, in a patient database.

The findings of whether there is a significant deviation from a reference value may be made in such a way that a relative deviation is determined during the course of treatment. This may be done in such a manner that a previously determined deviation is “calculated from” a reference value, and for the determination of whether there is a significant deviation, only a further deviation in comparison with a deviation already determined previously is taken into account.

If the deviation is significant, then in a sixth step S5 the cause of a deviation is detected as a function of the time determined. To do so, the time at which the dialysance or clearance was determined previously and thus also the time at which the deviation in the dialysance or clearance from the ideal course over time may be allocated to a certain treatment phase and the cause of the deviation from an ideal course of treatment may be recognized as a function of the certain treatment phase. In other words, the cause of a deviation from an ideal course of treatment may be allocated to a certain treatment phase. The allocation may also be done in a fuzzy manner, for example, by indicating probabilities for the corresponding causes. The cause of a deviation from an ideal course of treatment or an ideal operating state of the dialyzer should also be understood to include a certain group of possible causes, where the allocation in this case is made to a group of causes. The cause of a deviation from an ideal course of treatment may be displayed in an error message for the user, either as an independent error message or together with the error message indicating a deviation in the dialysance or clearance. Alternatively or additionally the cause of the deviation may be stored in a patient data record, for example, in a patient data record in a patient database.

One method of allocating a point in time in a treatment phase to a cause is described below in conjunction with FIG. 5.

FIG. 5 shows a method S5 for detecting the cause of a deviation from an ideal operating state of a blood treatment as a function of a point in time, at which the clearance or dialysance differs significantly from a reference value representing an ideal course of treatment or an ideal operating state of the dialyzer. The method S5 may be performed, for example, by the measurement and control device 25 from FIG. 1.

In a first step S51, a point in time to at which the previously determined value of the dialysance or clearance deviates from the reference value is determined.

The point in time may be determined by performing a plurality of dialysance measurements at regular points in time ti and comparing the measured value thereby obtained for the dialysance with a reference value corresponding to the respective point in time ti. If a significant deviation from the reference value is found, then the measurement point in time ti is allocated to a treatment phase.

As an alternative the dialysance at a predetermined point in time within each treatment phase may be measured so that an allocation between the measurement point in time and the treatment phase is predetermined.

In a second step S511 the point in time to is allocated to a certain treatment phase, namely in the present example a first, second or third treatment phase S52, S54 or S56.

The first treatment phase is typically at the start of the treatment. In an advantageous embodiment, the first treatment phase is the interval of time between the starting time of the treatment and 30 minutes after the starting time of the treatment.

If in a step S52 it is recognized that the measurement point in time ti lies in the first treatment phase, then in a step S53 a faulty positioning of the connectors of the dialyzer or a faulty positioning of the needles with respect to the direction of flow in the vascular access may be recognized as the cause of a deviation from an ideal course of treatment or an ideal operating state. These causes may be summarized as belonging to the group of operator-related causes for a deviation from an ideal course of treatment. If the faulty positioning of the needles or the vascular access is displayed to the operator by an error message at the start of the treatment, then at this early point in time of the treatment the operator still has the possibility of interrupting the treatment to correct the faulty positioning. If the classification of the dialysance or clearance as significant is to be made as a function of a percentage deviation, and the percentage is given as a function of the treatment phase, then a deviation of more than 10% above or below the predetermined reference value has proven expedient for the first treatment phase.

If the connectors of the dialyzer have been switched, the dialysate is operated in cocurrent instead of in countercurrent with the guidance of blood, as desired. The mathematical relationship for clearance is known for both cases (for example, J. Sargent and F. Gotch, “Principles and Biophysics of Dialysis” in J. Maher (editor), Replacement of Renal Function by Dialysis, Kluwer, 1989, 3^{rd} edition).

For example, starting from the mass transfer coefficient KoA, the flow rate of the dialysate in the dialyzer Q_{Di} and the blood-water flow rate in the dialyzer Q_{Di} in the case of hemofiltration and operation of the dialyzer in countercurrent, the following equation can be given for a theoretical estimate or a theoretical value K_{HDcounter} of the clearance:

$${K}_{HDcounter} ( k 0 A , {Q}_{B} , {Q}_{D} ) := {Q}_{B} \cdot \frac{exp [ k 0 A \cdot ( \frac{1}{{Q}_{B}} - \frac{1}{{Q}_{D}} ) ] - 1}{exp [ k 0 A \cdot ( \frac{1}{{Q}_{B}} - \frac{1}{{Q}_{D}} ) ] - \frac{{Q}_{B}}{{Q}_{D}}}$$

For the clearance K_{HDcocurr} of the same dialyzer at the flow rate of the dialysate in the dialyzer Q_{Di} and the blood-water flow rate in the dialyzer Q_{Bi}, the following equation holds for cocurrent operation:

$${K}_{HDcocurr} ( k 0 A , {Q}_{B} , {Q}_{D} ) := {Q}_{B} \cdot \frac{1 - exp [ - k 0 A \cdot ( \frac{1}{{Q}_{B}} - \frac{1}{{Q}_{D}} ) ]}{1 + \frac{{Q}_{B}}{{Q}_{D}}}$$

The expected standardized deviation Δ_{cocurrent} between the clearance can be calculated with a known k0A value of the filter, blood flow Q_{B} and dialysate flow Q_{D}:

$${Δ}_{cocurrent} ( k 0 A , {Q}_{B} , {Q}_{D} ) := 1 - \frac{{K}_{HDcocurr} ( k 0 A , {Q}_{B} , {Q}_{D} )}{{K}_{HDcounter} ( k 0 A , {Q}_{B} , {Q}_{D} )}$$

Thus, for example, for an FX80 dialyzer from the present applicant with a blood flow rate of 300 mL/min and a dialysate flow of 500 mL, the deviation is 32%, i.e., when the connectors are switched, the clearance is 32% lower than the value expected for countercurrent. The deviation is 24% at a Q_{D} of 800 mL/min with otherwise identical conditions. If the deviation between the measured clearance and the clearance expected for countercurrent is within the range expected for cocurrent, switching of the couplings of the dialyzer can be presumed to be the cause of the disturbance.

If it is recognized in a step S54 that the measurement point in time ti lies in the second treatment phase, then in a step S55 recirculation is recognized as the cause of the deviation from an ideal course of treatment or an ideal operating state.

Typically the second treatment phase is a middle treatment phase, i.e., a treatment phase in the middle of the treatment which comprises the period of time 60 minutes after the start of the treatment, for example, approximately 30-90 minutes after the start of the treatment, approx. 45-75 minutes after the start of the treatment or 55-65 minutes after the start of the treatment. The second treatment phase advantageously lies after a point in time at which a pressure-holding test has already been performed. If the classification of a dialysance or clearance as significant is to be made as a function of a percentage deviation, and the percentage is given as a function of the treatment phase, then a deviation of more than 15% above or below the predetermined treatment phase has proven expedient for the second treatment phase.

The second treatment phase may be selected so that the (relative) blood volume is close to the normohydration state and the aging of the set of blood tubes is in a range in which the blood flow indicated best corresponds to the actual blood flow. At this point in time of the treatment, typically clotting of the membrane has not yet occurred.

If a treatment phase is selected after the point in time when the pressure-holding test was performed, then an effect of the formation of secondary membranes can most likely be ruled out.

Since other causes for a deviation from an ideal course of treatment in this treatment phase can most likely also be ruled out, this treatment phase is especially suitable for detecting recirculation as the cause of a deviation from an ideal course of treatment.

A step 551 of triggering a recirculation measurement with a blood temperature monitor may precede step 55 of detecting that a recirculation is the cause of the deviation from an ideal operating state. In a recirculation measurement with a blood temperature monitor, first a change in the temperature of the blood in the venous branch of the extracorporeal blood circulation downstream from the dialyzer is triggered and then the temperature is measured in the arterial branch of the extracorporeal circulation upstream from the dialyzer. The change in temperature in the arterial branch due to a prior change in temperature in the venous branch can be used as a criterion for the determination of recirculation.

This recirculation measurement with the blood temperature monitor is thus a second measurement method which is independent of the measurement of clearance and can be used to confirm or not recirculation as the cause of a deviation from an ideal operating state of the blood treatment.

If it is detected in a step S56 that the measurement point in time ti lies in the third treatment phase, then in an advantageous embodiment, first an interim step S57 is performed in which a measurement of the attenuation of a pressure pulse perpendicular to the membrane is performed. To do so, the effect of a pressure source in the extracorporeal circulation, for example, the effect of pump on a pressure pickup in the extracorporeal circulation may be compared with the effect in the dialysis fluid circulation. To do so, as described in greater detail in conjunction with FIG. 1, the signal picked up by a pressure pickup in the dialysis fluid circulation may be broken down in its frequency spectrum and compared with the frequency spectrum of the signal picked up by the pressure pickup in the extracorporeal blood circulation.

If the attenuation measurement yields an increased attenuation of the pressure pulse in comparison with a reference value, then in a step S58 immediately after step S56 it is recognized that clotting of the semipermeable membrane of the dialyzer has occurred.

The third treatment phase typically extends from the end of the second treatment phase until the end of the treatment, i.e., approximately from 65 minutes after the start of the treatment until the end of the treatment, from 75 minutes after the start of the treatment until the end of the treatment, from 65 minutes after the start of the treatment until the end of the treatment or from 60 minutes after the start of the treatment until the end of the treatment.

The finding of whether there is a significant deviation from a reference value may advantageously be made in the case of the third treatment phase in such a manner that a relative deviation is determined during the course of treatment. This may be done in such a manner that a deviation from a reference value determined in the second treatment phase is “calculated out” to take into account an existing recirculation and for the determination of whether there is a significant deviation, only another deviation in comparison with a deviation already determined previously is taken into account. This additional deviation is understood to mean that it is attributable to clotting of the semipermeable membrane.

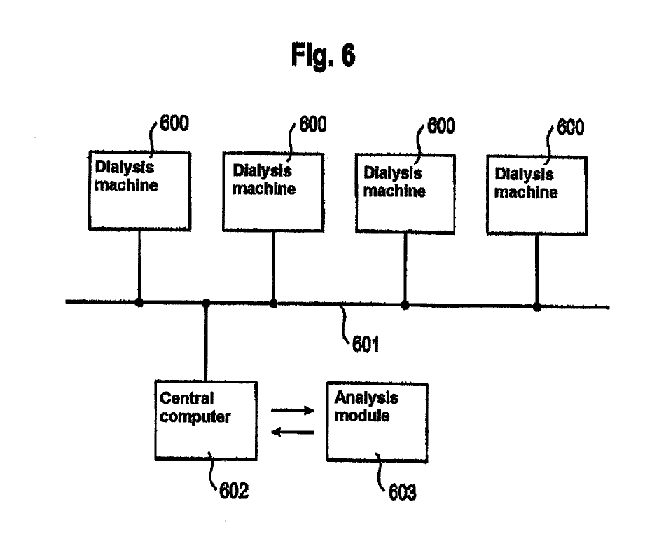

FIG. 6 shows a number of dialysis machines 600 which are linked together by a network 601 and are connected to a central computer 602. The dialysis machines 600 correspond in their design to the dialysis device 100 in FIG. 1. The central computer 602 is connected to an evaluation module 603, which comprises the functionality of the control and evaluation unit 25 of FIG. 1 or parts thereof. In particular the evaluation module 603 is suitable for controlling the actuators and sensors of the dialysis machines 600 and performing the method shown in FIG. 4 and/or the method shown in FIG. 5.

1. A device (25) for detecting the cause of a deviation from an ideal operating state or an ideal course of operation in an extracorporeal blood treatment, in which the blood to be treated in an extracorporeal blood circulation (9) flows through a blood chamber (3) of a dialyzer (1) subdivided by a semipermeable membrane (2) into the blood chamber (3) and a dialysis fluid chamber (4) and dialysis fluid in a dialysis fluid circulation (10) flows through the dialysis fluid chamber (4) of the dialyzer, comprising
Means for changing a physical or chemical characteristic (170; 171) of the dialysis fluid upstream from the dialysis fluid chamber (1) during the blood treatment, measurement means (174) for measuring a physical or chemical characteristic of the dialysis fluid downstream from the dialysis fluid chamber, an apparatus for determining a value of the dialysance or clearance from a change in the measured physical or chemical characteristic at a certain point in time during the blood treatment, an apparatus (25) for detecting a deviation in the certain value of the dialysance or clearance from a reference value of the dialysance or clearance, and an evaluation unit (25) for detecting the cause of a deviation from an ideal operating state as a function of the certain point in time.

2. The device according to claim 1, wherein the apparatus (25) for determining a deviation in the certain value of the dialysance or clearance from a reference value is designed to determine the reference values from a previous blood treatment or on the basis of a theoretical relationship.

3. The device according to claim 2, wherein the apparatus (25) for determining a deviation in the certain value of the dialysance or clearance from a reference value is designed to correct or compensate for the reference value using a current operating parameter of the blood treatment.

4. The device according to claim 1, wherein the evaluation unit (25) is adjusted to detect, as a function of a treatment point in time in a first treatment section, an incorrect positioning of the connectors of the dialyzer or an incorrect positioning of the needles in the extracorporeal circulation as the cause of the deviation from an ideal operating state.

5. The device according to claim 1, wherein the evaluation unit (25) is adjusted as a function of a treatment point in time in a second treatment section to detect recirculation as the cause of the deviation from an ideal operating state.

6. The device according to claim 5, wherein the evaluation unit is adjusted to trigger a change in the temperature of the blood in the extracorporeal blood circulation, to trigger a measurement of the temperature in the extracorporeal circulation upstream from the dialyzer, to use the measured temperature for determining a measure of the recirculation and to take it into account in detecting recirculation as the cause of the deviation.

7. The device according to claim 5, comprising means for determining a point in time at which a pressure holding test was performed, wherein the evaluation unit is adjusted, as a function of a point in time at which previously a pressure holding test was performed to detect recirculation as the cause of the deviation from an ideal operating state.

8. The device according to claim 1, wherein the evaluation unit is adjusted to detect recirculation as the cause of the deviation from an ideal operating state, after previously having ruled out incorrect positioning of the connectors of the dialyzer or incorrect positioning of the needles.

9. The device according to claim 1, wherein the evaluation unit is adjusted, as a function of a point in time in a third treatment step to detect clotting of the semipermeable membrane as the cause of the deviation from an ideal operating state.

10. The device according to claim 1, comprising measurement means (34; 33) for measuring a damping of a pressure pulse in the dialyzer (1), means for generating a control signal for triggering a measurement of the damping of the pressure pulse, when previously a deviation in the dialysance was determined, wherein the evaluation unit is adjusted to take into account a measured damping of the pressure pulse in detecting clotting of the semipermeable membrane.

11. A blood treatment device having a dialyzer (1) divided by a semipermeable membrane (2) into a blood chamber (3) and a dialysis fluid chamber (4), wherein the blood chamber (4) is connected to an extracorporeal blood circulation (9) and the dialysis fluid chamber (4) of the dialyzer (1) is connected to a Dialysis fluid circulation(10) and a device for detecting the cause of a deviation from an ideal operating state or an ideal course of operation of an extracorporeal blood treatment according to claim 1.

12. A method for detecting the cause of a deviation from an ideal operating state or an ideal course of operation in an extracorporeal blood treatment in which the blood to be treated in an extracorporeal blood circulation (9) flows through a blood chamber of a dialyzer (1), which is subdivided by a semipermeable membrane (2) into the blood chamber (3) and a dialysis fluid chamber (4), and dialysis fluid flows through the dialysis fluid chamber (4) of the dialyzer (1) in a dialysis fluid circulation (10), comprising the following steps:
Changing a physical or chemical variable of the dialysis fluid upstream from the dialysis fluid chamber during the blood treatment (S1) and
Measuring a physical or chemical characteristic of the dialysis fluid downstream from the dialysis fluid chamber (S2),
Determining a value of the dialysance or clearance from a change in the measured physical or chemical characteristic at a certain point in time during the blood treatment (S3),
Determining a deviation in the certain value of the dialysance or clearance from a reference value of the dialysance or clearance (S4), and
Detecting the cause of a deviation from an ideal operating state of the blood treatment as a function of the certain point in time (S5).

13. The method according to claim 12, wherein the reference value is determined from a previous blood treatment or on the basis of a theoretical relationship.

14. The method according to claim 13, wherein the reference value of a prior treatment is corrected or compensated using a current operating parameter of the blood treatment.

15. The method according to claim 12, wherein the certain point in time at which the deviation in the value of the dialysance or clearance is determined is in a first treatment section, and an incorrect positioning of the connectors of the dialyzer or an incorrect positioning of the needles is detected as the cause of the deviation from an ideal operating state (S53).

16. The method according to claim 12, wherein the certain point in time at which the deviation in the value of the dialysance or clearance is determined is in a second treatment section, and recirculation is recognized as the cause of the deviation from an ideal operating state (S55).

17. The method according to claim 16, wherein a change in the temperature of the blood in the extracorporeal blood circulation is triggered, a measurement of the temperature in the extracorporeal circulation upstream from the dialyzer is triggered, the measured temperature is used to determine a criterion of the recirculation, and the criterion of recirculation thereby determined is taken into account in detecting recirculation as the cause of the deviation.

18. The method according to claim 16, wherein the point in time at which recirculation is detected as the cause of the deviation from an ideal operating state is the point in time at which a pressure holding test was previously performed.

19. The method according to claim 16, wherein recirculation is detected as the cause of the deviation from an ideal operating state, after having previously ruled out incorrect positioning of the connectors of the dialyzer or incorrect positioning of the needles.

20. The method according to claim 11, wherein the certain point in time at which the deviation in the value of the dialysance or clearance is determined lies in a third treatment section, and clotting of the semipermeable membrane is recognized (S58) as the cause of the deviation from an ideal operating state.

21. The method according to claim 20, wherein after a deviation in the certain value of the dialysance or clearance from a reference value of the dialysance or clearance was determined, damping of a pressure pulse in the dialyzer is measured, and the measured damping of the pressure pulse in detecting clotting of the semipermeable membrane is taken into account.

22. A computer program product containing parts of program code adapted for executing a method according to claim 12.
